(19) United States
(12) Patent Application Publication
(10) Pub. No.: US 2011/0144040 A1
(43) Pub. Date: Jun. 16, 2011

(54) Acteoside and acteoside-rich plant extracts for increasing athletic performance in humans

(75) Inventors: Shawn Wells, Monroe, NC (US); William C. Llewellyn, Palm Beach Gardens, FL (US)

(73) Assignee: MN Intellectual Property Corp, LLC

(21) Appl. No.: 12/928,276

(22) Filed: Dec. 8, 2010

Related U.S. Application Data
(60) Provisional application No. 61/283,953, filed on Dec. 10, 2009.

(51) Int. Cl. A61K 31/7034; A61P 29/00; A61P 31/00; A23L 1/30

(52) U.S. Cl. 514/25; 426/648; 426/2

(57) ABSTRACT

The present invention relates to increasing the athletic performance of humans using acteoside. This may be in the form of a pure compound, or plant extracts containing at least 10% acteoside by weight. Application of this invention provides increased strength, muscle power, endurance, muscle protein content, and reduced fatigue.

### CROSS-REFERENCE TO RELATED APPLICATIONS

This patent application claims the benefit of provisional patent application 61/283,953, filed on Dec. 10, 2009.

### STATEMENT REGARDING FEDERALLY SPONSORED RESEARCH OR DEVELOPMENT

Not Applicable

### REFERENCE TO A MICROFICHE APPENDIX

Not Applicable

### BACKGROUND OF THE INVENTION

In recent years there has been growing interest in identifying natural medicines and plant extracts that can be used to improve athletic performance or body composition (the ratio of lean to fat mass) in humans. Traditional Chinese and Ayurvedic healing practices have been good sources for identifying plants of notable medicinal value, as many have been used for centuries to treat disease. In an isolated number of instances, traditional medicine protocols and extracts have been further investigated and modified by modern scientists, and made useful for improving the performance of athletes. Given the limited progress in this area, however, these inventors set out to further explore, identify, and develop strategies for more effective herbal substances for improving athletic performance.

Acteoside is a natural substance found in the plants rehmannia glutinosa, cistanche tubulosa, cistanche deserticola, cistanche salsa, abeliophyllum distichum, rosa rugosa, acanthus ilicifolius, callicarpa dichotoma, scrophularia ningpoensis, ligstrum purpurascens, stachys sieboldii, buddleja globosa, nepeta ucrainica, lantana camara, scutellaria baicalensis, and verbena littoralis. These plant species have a long history of use treating disease in traditional Ayurvedic and Chinese medicine. These plants also have medicinal properties supported by the modern medical literature. In spite of their widespread use and study, however, acteoside (itself or in the form of extracts containing at least 10% of this substance by weight) has not been explored as a potential medicine or health food supplement for improving athletic performance in humans. After extensive research and investigation, these inventors have developed a novel use for acteoside and acteoside-rich plant extracts. When properly applied, they improve muscle protein content and athletic performance in human athletes and other active exercising individuals.

### BRIEF SUMMARY OF THE INVENTION

The present invention involves methods for utilizing, and compositions containing, acteoside for improving athletic performance in humans. The acteoside is given orally to an athlete or exercising individual, in capsule, pill, powder, edible bar, or liquid form. The acteoside may be a purified compound, or an extract of rehmannia glutinosa, cistanche tubulosa, cistanche deserticola, cistanche salsa, abeliophyllum distichum, rosa rugosa, acanthus ilicifolius, callicarpa dichotoma, scrophularia ningpoensis, ligstrum purpurascens, stachys sieboldii, buddleja globosa, nepeta ucrainica, lantana camara, scutellaria baicalensis, or verbena littoralis, or a combination thereof, containing at least 10% acteoside by weight. The acteoside is given in an oral dose ranging from between 25 mg and 2,500 mg. This dose is repeated daily, or on a regular schedule consistent with training (such as in conjunction with training days if daily use is not preferred). For most notable results, the extract is given for a minimum period of 2-3 weeks. When taken consistently in such a manner, and when its use is accompanied by regular athletic competition, exercise, or high physical activity, acteoside can significantly improve athletic performance. This is seen by improvements in such variables as running speed, endurance, muscle strength, and/or muscle protein content. Such improvements may be noticed either during athletic competition, or during/following exercise in support of such activity. In addition, the ratio of lean muscle mass to fat mass may be increased in the body as a result of supplementing with acteoside.

### BRIEF DESCRIPTION OF THE SEVERAL VIEWS OF THE DRAWING

Not Applicable

### DETAILED DESCRIPTION OF THE INVENTION

Rehmannia glutinosa is an herbal plant native to China, Japan and Korea. It is commonly called “Chinese Foxglove”. Rehmannia glutinosa has a long history of use in traditional Chinese medicine, where it has been applied to treat such conditions as treat rheumatoid arthritis, asthma, urticaria, and chronic nephritis. Cistanche tubulosa is another herbal medicinal plant native to Asia. It is often referred to as “desert ginseng”. It has been widely used in the treatment of many conditions including male impotency, infertility, metrorrhagia, vaginal discharge, osteoporosis, physical weakness, and constipation. These medicinal plants, as well as others including cistanche deserticola, cistanche salsa, abeliophyllum distichum, rosa rugosa, acanthus ilicifolius, callicarpa dichotoma, scrophularia ningpoensis, ligstrum purpurascens, stachys sieboldii, buddleja globosa, nepeta ucrainica, lantana camara, scutellaria baicalensis, and verbena littoralis are natural sources of acteoside. This invention relates to the use of acteoside and plant extracts containing at least 10% acteoside by weight to improve athletic performance in humans.

Acteoside is a naturally occurring phenylpropanoid glycosidic compound. It is found in a variety of plant species, and is believed to have several distinct biologically active effects. The most widely documented activities of acteoside appear to be that of a strong inhibitor of both prostaglandin biosynthesis (Arch Pharm Res 2006 29(6), 508-13) and free-radical induced oxidative stress (J Pharm Pharmacol 2004 56(6) 743-8). Thus, the compound appears to be both an anti-inflammatory and an antioxidant. Studies also suggest acteoside has anti-estrogenic (J Steroid Biochem Mol Biol 98(2006) 63-71), liver protecting (Life Sciences 2004 74(8), 1051-64), memory and cognition improving/protecting (Biol. Pharm. Bull 29(1) 71-74,2006), and immunomodulatory (J Pharm Pharmacol 2006 58(9), 1275-80) properties. While it is well understood that acteoside and acteoside-containing plant extracts possesses a wide spectrum of medicinal/biologically active properties, a full understanding of these properties and their underlying mechanisms of action is still lacking.

Prior to the work of these inventors, no investigations into the effects of acteoside or acteoside-rich plant extracts on athletic performance or muscle protein content in humans have taken place. There has also been no unsupported suggestion or theory in the medical literature that ergogenic activity may be present in acteoside or acteoside-containing plant extracts, nor any mention of such attempted use. The use of acteoside or acteoside-rich plant extracts according to this invention came only after a long effort to research Ayurvedic and traditional Chinese medical preparations, and discover what, and under what context, particular plant species can be used to improve the results of exercise and athletic performance. Acteoside was shown to possess unique beneficial activities in this regard, according to these inventors.

It is the object of this invention to improve athletic performance by the oral administration of acteoside and acteoside-rich extracts of rehmannia glutinosa, cistanche tubulosa, cistanche deserticola, cistanche salsa, abeliophyllum distichum, rosa rugosa, acanthus ilicifolius, callicarpa dichotoma, scrophularia ningpoensis, ligstrum purpurascens, stachys sieboldii, buddleja globosa, nepeta ucrainica, lantana camara, scutellaria baicalensis, and/or verbena littoralis. The extracts used for the purpose of this invention may have been obtained with any of a variety of standard extraction methods. These typically involve the blending of dried plant material with one or more liquid extraction agents. The extraction agent is later separated from the leftover plant material, and then dried, heated, evaporated, distilled, or otherwise removed from the mixture. This will yield a powdered extract that contains medically active plant molecules. The subject of this invention is acteoside or extracts containing at least 10% acteoside by weight, which were obtained with at least one extraction agent selected from the group consisting of water, alcohol, chloroform, petrol ether, dichloromethane, acetic acid, liqueur wine, and a supercritical liquid. When said extraction agent is an alcohol, at least one alcohol selected from the group consisting of methanol, ethanol, propanol, butanol, pentanol and hexanol may be used.

Athletic performance is defined as any sporting activity where muscle strength and/or endurance are integral to individual performance, and is inclusive of both aerobic and anaerobic metabolic conditions. This includes active athletic competition, such as baseball, football, running, or Track & Field. It also includes any exercise activity designed to improve physical performance, directly or indirectly, via an improvement in muscle protein content (size), strength, and/or endurance, such as resistance training (circuit equipment, weight training), aerobics, Pilates, and/or calisthenics. Any improvement in athletic performance must include objective increases in speed, strength, muscle size (protein content), and/or endurance. It may also involve improved recovery from fatigue following exercise or athletic competition.

Surprisingly, it was shown during our studies that acteoside and acteoside-rich extracts act as effective in-vivo peroral agents for improving athletic performance when used according to our recommended protocols. We found acteoside to possess a wide spectrum of performance-enhancing activity, in fact. This includes an ability to increase muscle strength, muscle power, endurance, and muscle protein content (size), as well as to reduce fatigue and facilitate recovery following intense exercise or athletic competition. The benefits appear almost immediately in some cases, but are more likely to occur after several weeks of regular use of acteoside or extracts containing acteoside. The benefits were apparent in such level that it appears highly likely that acteoside and acteoside-rich extracts can have a very strong influence over the exercise and during-competition performance of supplemented athletes.

The present invention provides methods of using pharmaceutical and dietary health supplement compositions of the inventive compound. Such compositions may be for oral administration. The treatment may consist of a single dose or a plurality of doses over a period of time. In general, comprehended by the invention are compositions comprising effective amounts of a compound of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 90), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hyaluronic acid may also be used, and this may have the effect of promoting sustained duration in the circulation. The pharmaceutical and dietary health supplement compositions optionally may include still other pharmaceutically acceptable liquid, semisolid, or solid diluents that serve as pharmaceutical vehicles, excipients, or media, including but are not limited to, polyoxyethylene sorbitan monolaurate, magnesium stearate, methyl- and propylhydroxybenzoate, starches, sucrose, dextrose, gum acacia, calcium phosphate, mineral oil, cocoa butter, and oil of theobroma. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435 1712 which are herein incorporated by reference. The compositions may be prepared in liquid form, or may be in dried powder, such as lyophilized form. Sustained release formulations are also contemplated, as are transdermal formulations.

Contemplated for use herein are oral solid dosage forms, which are described generally in Remington's Pharmaceutical Sciences, 18th Ed. 1990 (Mack Publishing Co. Easton Pa. 18042) at Chapter 89, which is herein incorporated by reference. Solid dosage forms include tablets, capsules, pills, troches or lozenges, cachets or pellets, and powder that may or may not be dissolved in a liquid. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the acteoside or acteoside-rich plant extract is illustrated by Marshall, K., Modern Pharmaceutics, Edited by G. S. Banker and C. T. Rhodes Chapter 10, 1979, herein incorporated by reference. In general, the formulation will include the inventive compound, and inert ingredients which allow for protection against the stomach environment, and release of the biologically active material in the intestine. The oral dosage forms contemplated herein can be administered at any time of the day or night. These oral dosage forms may be administered or taken one or more times per day or night. It is preferable that the oral dosage forms be administered or taken between 1 and 10 times per day or night. It is still more preferable that the oral dosage forms be administered or taken between 1 and 5 times per day or night. It is still more preferable that the oral dosage forms be administered or taken between 2 and 3 times per day or night.

Also specifically contemplated are oral dosage forms of the above inventive compounds. If necessary, the compounds may be chemically modified so that oral delivery is efficacious. Generally, the chemical modification contemplated is the attachment of at least one moiety to an active molecule itself, where said moiety permits (a) inhibition of proteolysis; and (b) uptake into the blood stream from the stomach or intestine. Also desired is the increase in overall stability of the compound and increase in circulation time in the body. Examples of such moieties include: Polyethylene glycol, copolymers of ethylene glycol and propylene glycol, carboxymethyl cellulose, dextran, polyvinyl alcohol, polyvinyl pyrrolidone and polyproline (Abuchowski and Davis, Soluble Polymer-Enzyme Adducts, Enzymes as Drugs, Hocenberg and Roberts, eds., Wiley-Interscience, New York, N.Y., (1981), pp 367 383; Newmark, et al., J. Appl. Biochem. 4:185 189 (1982)). Other polymers that could be used are poly-1,3-dioxolane and poly-1,3,6-tioxocane. Preferred for pharmaceutical usage, as indicated above, are polyethylene glycol moieties.

For the oral delivery dosage forms, it is also possible to use a salt of a modified aliphatic amino acid, such as sodium N-(8-[2-hydroxybenzoyl]amino) caprylate (SNAC), as a carrier to enhance absorption of the compounds of this invention. The clinical efficacy of a heparin formulation using SNAC has been demonstrated in a Phase II trial conducted by Emisphere Technologies. See U.S. Pat. No. 5,792,451, “Oral drug delivery composition and methods”.

The acteoside or acteoside-rich plant extract can be included in the formulation as fine multiparticulates in the form of granules or pellets of particle size about 1 mm. The formulation of the material for capsule administration could also be as a powder, lightly compressed plugs or even as tablets. The compound could be prepared by compression.

Colorants and flavoring agents may all be included. For example, the protein (or derivative) may be formulated (such as by liposome or microsphere encapsulation) and then further contained within an edible product, such as a refrigerated beverage containing colorants and flavoring agents.

One may dilute or increase the volume of the acteoside or acteoside-rich plant extract with an inert material. These diluents could include carbohydrates, especially mannitol, .alpha.-lactose, anhydrous lactose, cellulose, sucrose, modified dextrans and starch.

Certain inorganic salts may also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of acteoside or acteoside-rich plant extract into a solid dosage form. Materials used as disintegrants include but are not limited to starch including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants is the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the acteoside or acteoside-rich plant extract together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the contemplated compounds.

Controlled release formulation may be desirable. The contemplated compounds could be incorporated into an inert matrix which permits release by either diffusion or leaching mechanisms e.g., gums. Slowly degenerating matrices may also be incorporated into the formulation, e.g., alginates, polysaccharides. Another form of a controlled release of the contemplated compounds is by a method, wherein the compound is enclosed in a semipermeable membrane which allows water to enter and push the compounds out through a single small opening due to osmotic effects. Some enteric coatings also have a delayed release effect.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto The following non-limiting example and data illustrates various aspects and features relating the methods and compositions of the present invention. While the utility of this invention may be illustrated through the use of several methods and compositions, it will be understood by those skilled in the art that comparable results are obtainable with various other methods, as are commensurate with the scope of this invention.

### EXAMPLE I

### Effective Dosages

During the course of developing this invention, an effective oral daily dosage of acteoside to improve athletic performance was determined to be between 25 mg to 2,500 mg. The total daily dosage can be further subdivided for more sustained blood concentrations of acteoside, with 2-3 applications per day being most preferred. The preferred embodiment of this invention involves the daily oral use of acteoside, and/or acteoside-rich plant extract(s), which is continued for a minimum of 2-3 weeks. The supplementation accompanies an athletic competition or exercise program, or active lifestyle with significant physical activity.

We claim:

1. A method of increasing athletic performance in humans, which comprises administering orally an effective amount of acteoside.

2. A composition to be administered to humans for increasing athletic performance, which comprises an effective amount of acteoside.

3. A medicine or dietary health supplement comprising the composition of claim 2 in capsule, pill, powder, edible bar, or liquid forms.

4. A composition of claim 2, comprising an extract from rehmannia glutinosa, cistanche tubulosa, cistanche deserticola, cistanche salsa, abeliophyllum distichum, rosa rugosa, acanthus ilicifolius, callicarpa dichotoma, scrophularia ningpoensis, ligstrum purpurascens, stachys sieboldii, buddleja globosa, nepeta ucrainica, lantana camara, scutellaria baicalensis, or verbena littoralis, or a combination thereof, wherein said extract(s) contain(s) not less than 10% acteoside by weight.

5. A composition of claim 2, wherein the extract is obtained with at least one component selected from the group consisting of water, alcohol, chloroform, petrol ether, dichloromethane, acetic acid, liqueur wine, and a supercritical liquid as an extraction agent.

6. A composition of claim 5, wherein said extraction agent is at least one alcohol selected from the group consisting of methanol, ethanol, propanol, butanol, pentanol and hexanol.
